(19) United States
(12) Patent Application Publication
(10) Pub. No.: US 2014/0251809 A1
(43) Pub. Date: Sep. 11, 2014

(54) APPARATUS FOR PREPARING NUCLEIC ACIDS AND METHOD FOR PREPARING NUCLEIC ACIDS

(71) Applicant: Sony Corporation, Tokyo (JP)

(72) Inventors: Naohisa Sakamoto, Tokyo (JP); Tomohiko Nakamura, Tokyo (JP); Kazumine Ito, Tokyo (JP); Tasuku Yotoriyama, Tokyo (JP)

(73) Assignee: Sony Corporation, Tokyo (JP)

(21) Appl. No.: 14/182,603

(22) Filed: Feb. 18, 2014

(30) Foreign Application Priority Data
Mar. 8, 2013 (JP) 2013-047143

(51) Int. Cl. G01N 27/447; C12Q 1/68

(52) U.S. Cl. CPC G01N 27/44756; C12Q 1/6806
USPC 204/465; 204/615

(57) ABSTRACT

An apparatus for preparing nucleic acids is provided which includes an anode, a cathode, and a space formed between the anode and the cathode. In addition, in the apparatus described above, the space includes a first porous film provided at an anode side, a second porous film provided at a cathode side, an inlet port which is proved in a region sandwiched between the first porous film and the second porous film and which introduces a specimen containing proteins and nucleic acids into the region, and a gel filter provided between the inlet port and the first porous film.

### CROSS REFERENCE TO RELATED APPLICATIONS

This application claims the benefit of Japanese Priority Patent Application JP 2013-047143 filed Mar. 8, 2013, the entire contents of which are incorporated herein by reference.

### BACKGROUND

The present disclosure relates to an apparatus for preparing nucleic acids and a method for preparing nucleic acids. In more particular, the present disclosure relates, for example, to an apparatus for preparing nucleic acids, the apparatus including a gel filter in a space sandwiched between electrodes.

A method for analyzing nucleic acids has been used for a nucleic acid test to diagnose infectious diseases, detection of a single nucleotide polymorphism, and the like. In the analysis of nucleic acids as described above, in order to prevent components contained in a specimen other than the nucleic acids from adversely influencing the analysis, the content of the components contained in the specimen other than the nucleic acids is preferably decreased in many cases. Hence, in the analysis of nucleic acids, a method for preparing a specimen containing nucleic acids beforehand in a state suitable for analysis thereof has been desired.

For example, Japanese Unexamined Patent Application Publication No. 2005-95003 has disclosed a method in which in order to isolate nucleic acids contained in a specimen, the nucleic acids therein are adsorbed to a nucleic-acid adsorptive porous film. The method described above includes the steps of: passing a specimen solution containing nucleic acids through a nucleic-acid adsorptive porous film to adsorb the nucleic acids therein, passing a washing liquid through the nucleic-acid adsorptive porous film to wash the nucleic-acid adsorptive porous film together with the nucleic acids adsorbed therein, and passing a recovery liquid through the nucleic-acid adsorptive porous film to desorb the nucleic acids from the inside of the nucleic-acid adsorptive porous film.

### SUMMARY

By the isolation and refinement method for nucleic acids disclosed in Japanese Unexamined Patent Application Publication No. 2005-95003, the nucleic acids may be refined to a level suitable for analysis thereof. However, in the preparation method using a nucleic-acid adsorptive porous film described above, in all the adsorption, washing, and recovery steps, the liquids used in the respective steps are inevitably passed through the nucleic-acid adsorptive porous film, and hence a process for preparing nucleic acids is complicated.

Accordingly, it is primarily desirable to provide a method for easily preparing a specimen containing nucleic acids in a state suitable for analysis thereof without performing a complicated process.

According to an embodiment of the present disclosure, there is provided an apparatus for preparing nucleic acids including an anode, a cathode, and a space formed between the anode and cathode, and the space described above includes a first porous film provided at an anode side, a second porous film provided at a cathode side, an inlet port which is provided in a region sandwiched between the first porous film and the second porous film and which introduces a specimen containing proteins and nucleic acids into the region described above, and a gel filter provided between the inlet port and the first porous film.

The volume of a first region defined by the gel filter and the first porous film may be smaller than the volume of a second region defined by the gel filter and the second porous film.

In addition, an average pore diameter of the first porous film may be 1 to 10 nm, and the gel filter may be prepared to have a pH of 7 to 10.

Furthermore, the apparatus may further include a detection portion which detects a transfer of the proteins to a boundary of the gel filter to the first region.

The apparatus for preparing nucleic acids may be used to prepare a template nucleic acid of a nucleic acid amplification reaction.

In addition, besides the cathode described above, another cathode may be further provided in the gel filter.

As the cathode in the gel filter, a plurality of cathodes may be provided along a transfer direction of the nucleic acids.

Furthermore, a current application to the anode and the cathode in the gel filter may be switched to and from a current application to the anode and the cathode provided outside the gel filter.

In addition, according to an embodiment of the present disclosure, there is provided a method for preparing nucleic acids including the steps of: receiving a specimen containing proteins and nucleic acids in a region sandwiched between a cathode and a gel filter, the region being located in a space which is formed between the cathode and an anode and in which the gel filter is provided, applying a current to the anode and the cathode, and allowing the nucleic acids to migrate to a region sandwiched between the anode and the gel filter through the gel filter.

Furthermore, according to an embodiment of the present disclosure, there is also provided a method for preparing nucleic acids including the steps of: receiving a specimen containing proteins and nucleic acids in a region sandwiched between a cathode and a gel filter, the region being located in a space which is formed between the cathode and an anode and in which the gel filter is provided, applying a current to the anode and the cathode, and recovering the nucleic acids from a region sandwiched between the anode and the gel filter.

The gel filter may be prepared to have a pH of 7 to 10 for the use.

Before the receiving step is performed, a step of desalting the specimen may be further included, and a step of performing an ultrasonic treatment on the specimen may also be further included.

The method for preparing nucleic acids may be used as a method for preparing a template nucleic acid of a nucleic acid amplification reaction.

In addition, the nucleic acid amplification method may be performed by a polymerase chain reaction (PCR) method, and the nucleic acid amplification method may be an isothermal nucleic acid amplification reaction.

According to an embodiment of the present disclosure, for example, there is provided an apparatus for preparing a specimen containing nucleic acids in a state suitable for analysis thereof by a simple procedure.

### BRIEF DESCRIPTION OF THE DRAWINGS

FIG. 1 is a schematic cross-sectional view of an apparatus for preparing nucleic acids according to a first embodiment of the present disclosure;

FIG. 2 is a flowchart showing a method for preparing nucleic acids according to an embodiment of the present disclosure;

FIGS. 3A to 3C are schematic cross-sectional views each illustrating a preparation of nucleic acids using the apparatus for preparing nucleic acids according to the first embodiment;

FIG. 4 is a schematic cross-sectional view according to a modification of the first embodiment;

FIG. 5 is a schematic cross-sectional view of an apparatus for preparing nucleic acids according to a second embodiment of the present disclosure;

FIG. 6 is a schematic cross-sectional view of an apparatus for preparing nucleic acids according to a modification of the second embodiment;

FIG. 7 is a graph showing the result of a preparation of a specimen using the apparatus for preparing nucleic acids according to the first embodiment of the present disclosure (Experimental Example 1); and

FIG. 8 is a graph showing the result of a nucleic acid amplification reaction performed using nucleic acids prepared by the apparatus for preparing nucleic acids according to the first embodiment of the present disclosure (Experimental Example 2).

### DETAILED DESCRIPTION OF EMBODIMENTS

Hereinafter, preferable embodiments carrying out the present disclosure will be described. The following embodiments will be described as representative embodiments of the present disclosure, and hence it is not to be construed that the scope of the present disclosure is narrowed by the following embodiments. In addition, description will be made in the following order.

(1) An object of a method for preparing nucleic acids according to an embodiment of the present disclosure.
(2) An apparatus for preparing nucleic acids according to a first embodiment of the present disclosure.
(3) A method for preparing nucleic acids according to an embodiment of the present disclosure.
(4) An apparatus for preparing nucleic acids according to a modification of the first embodiment.
(5) An apparatus for preparing nucleic acids according to a second embodiment of the present disclosure.
(6) An apparatus for preparing nucleic acids according to a modification of the second embodiment.

### (1) An Object of a Method for Preparing Nucleic Acids According to an Embodiment of the Present Disclosure.

In a preparation of nucleic acids using an apparatus (hereinafter simply referred to as “preparation apparatus” in some cases) for preparing nucleic acids according to an embodiment of the present disclosure, a specimen containing nucleic acids and proteins is prepared in a state suitable for analysis of the nucleic acids.

The specimen is not particularly limited, and any specimens containing nucleic acids and proteins derived from animals, plants, fungi, bacteria, viruses, and the like may be used. As the nucleic acid, either a single or a double stranded nucleic acid may be used, and either DNA or RNA may also be used. In addition, the molecular weight of the nucleic acid is also not particularly limited. Incidentally, unlike a bacterial genome present in a bacterial cell, the nucleic acids contained in the specimen may be not to be directly dispersed in the specimen and may be enclosed with a membrane, such as a cell membrane, or may be present in a particle.

As the specimen containing nucleic acids, for example, there may be mentioned an organism-derived specimen. As the organism-derived specimen, for example, whole blood, blood plasma, blood serum, cerebral spinal fluid, urine, semen, swabs (such as nasal swab, throat swab, snivel, and phlegm) may be mentioned. In addition, diluted solutions of those organism-derived specimens are also categorized as the specimen to be prepared by the apparatus for preparing nucleic acids according to an embodiment of the present disclosure.

The analysis of nucleic acids is to analyze the feature and the amount of nucleic acids contained in a specimen by existing techniques. The analysis of the feature and the amount of nucleic acids includes various types of analyses, such as determination of base sequence of a nucleic acid chain, determination of a single nucleotide polymorphism, and quantitative determination of nucleic acids. As analysis methods, for example, a nucleic acid amplification method, a melting curve analysis method, a quantitative PCR method, a DNA array, and an RNA array may be mentioned. The apparatus and the method for preparing nucleic acids according to an embodiment of the present disclosure may be used to prepare a template nucleic acid of a nucleic acid amplification reaction.

As the nucleic acid amplification method, for example, a polymerase chain reaction (PCR) method which performs a temperature cycle is preferable. In addition, as the nucleic acid amplification reaction, various types of isothermal amplification methods, each of which performs no temperature cycle, may also be used. As the isothermal amplification method, for example, a loop-mediated isothermal amplification (LAMP) method and a transcription-reverse transcription concerted (TRC) method may be mentioned. The method for preparing a specimen for a nucleic acid amplification reaction according to an embodiment of the present disclosure is also suitably used for a method for amplifying nucleic acids by an isothermal amplification reaction, and as the isothermal amplification method, for example, a LAMP method is preferable.

### (2) An Apparatus for Preparing Nucleic Acids According to a First Embodiment of the Present Disclosure.

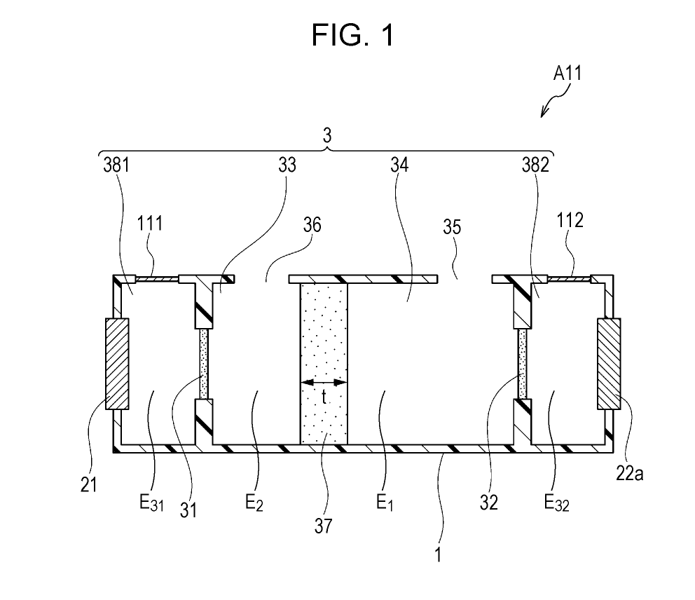

FIG. 1 is a schematic cross-sectional view of an apparatus (hereinafter simply referred to as “preparation apparatus” in some cases) for preparing nucleic acids according to a first embodiment of the present disclosure. In FIG. 1, a preparation apparatus designated by reference numeral A11 includes an anode 21, a cathode 22a, and a space 3 formed between the anode 21 and the cathode 22a. In addition, this space 3 includes a first porous film 31 provided at an anode 21 side, a second porous film 32 provided at a cathode 22a side, an inlet port 35 which is provided in a region sandwiched between the first porous film 31 and the second porous film 32 and which introduces a specimen containing proteins and nucleic acids into the region described above, and a gel filter 37 provided between the inlet port 35 and the first porous film 31. With reference to FIG. 1, constituent elements of the preparation apparatus A11 will be described.

### <Space>

In the preparation apparatus A11, the space 3 is formed between the anode 21 and cathode 22a. In the preparation apparatus A11 shown in FIG. 1 as an example, the space 3 corresponds to an introduction portion 34 (second region), a recovery portion 33 (first region), and buffer solution receiving portions 381 and 382. In addition, in the preparation apparatus A11, the space 3 is held between the anode 21 and the cathode 22a and is surrounded by a housing 1 so that the specimen and the like are received in the space 3.

The housing 1 may be formed from a material having insulating properties and resistance against, for example, heat generated by a current application to the anode 21 and the cathode 22a which will be described later, and the material is not particularly limited. As the material of the housing 1, for example, common plastic materials may be used. As the plastic materials, for example, a polyethylene (PE), a polystyrene (PS), a polypropylene (PP), and an acrylic resin (such as a poly(methyl methacrylate) (PMMA)) may be mentioned.

In the space 3 shown in FIG. 1, the first porous film 31, the second porous film 32, and the gel filter 37 are provided. Accordingly, the space 3 is divided into the buffer solution receiving portion 381 located between the anode 21 and the first porous film 31, the first region (recovery portion) 33 located between the first porous film 31 and the gel filter 37, the second region (introduction portion) 34 located between the gel filter 37 and the second porous film 32, and the buffer solution receiving portion 382 located between the second porous film 32 and the cathode 22a.

The first porous film 31, the second porous film 32, and the gel filter 37 will be described later. In addition, in description of the preparation apparatus A11, for the sake of convenience, the first region 33 and the second region 34 are called the recovery portion 33 and the introduction portion 34, respectively, in accordance with the functions of the respective regions in the preparation apparatus A11.

The introduction portion 34 provided in the space 3 is a space E₁ into which a specimen containing proteins and nucleic acids is to be introduced, and the introduction portion 34 communicates with the outside of the preparation apparatus A11 through the inlet port 35.

The recovery portion 33 is a space E₂ which the nucleic acids contained in the specimen reach after passing through the gel filter 37 and is a region from which the nucleic acids are recovered. In addition, a recovery port 36 is provided to transfer a liquid containing the nucleic acids in the recovery portion 33 to another container or the like. In addition, by the reason which will be described later, the volume of the recovery portion 33 (first region) defined by the gel filter 37 and the first porous film 31 is preferably small as compared to the volume of the introduction portion 34 (second region) defined by the gel filter 37 and the second porous film 32.

The buffer solution receiving portions 381 and 382 are spaces E₃₁ and E₃₂, respectively, each of which receives a buffer solution. In addition, those buffer solutions in the spaces E₃₁ and E₃₂ are in contact with the respective electrodes (the anode 21 and the cathode 22a) which will be described later. In the preparation apparatus A11 according to the first embodiment of the present disclosure, the compositions of the buffer solutions received in the buffer solution receiving portions 381 and 382 may be appropriately prepared in accordance with the composition of the gel filter 37 and the like. In addition, when a buffer solution prepared to have a pH approximately equivalent to that of the gel filter 37 is received in the buffer solution receiving portions 381 and 382, in the electrophoresis which will be described later, the pH of the gel filter 37 may be stabilized.

As the buffer solution, for example, 1×TAE (0.04 M Tris, 0.04 M acetate, 0.001 M EDTA) or 1×TBE (0.089 M Tris-borate, 0.089 M Boric Acid, 0.002 M EDTA) may be used.

In addition, by a current application to the anode 21 and the cathode 22a in a method for preparing nucleic acids according to an embodiment of the present disclosure which will be described later, gases are generated in the buffer solution receiving portions 381 and 382. Hence, in order to discharge those gases outside the preparation apparatus A11, the spaces E₃₁ and E₃₂ of the buffer solution receiving portions 381 and 382, respectively, are preferably not to be tightly sealed. For example, the buffer solution receiving portions 381 and 382 may be each provided with an opening. When the openings are provided, in order to discharge the generated gases and to retain the buffer solutions, which are liquids, in the buffer solution receiving portions 381 and 382, gas permeable films 111 and 112 are preferably provided for the openings.

### <Gel Filter>

In the preparation apparatus A11 according to the first embodiment of the present disclosure, the gel filter 37 is configured to separate nucleic acids from proteins and to decrease the concentration of proteins in the recovery portion 33 as compared to that of proteins in the introduction portion 34. The separation of nucleic acids from proteins using the gel filter 37 will be described later.

The gel filter 37 may be configured so as to enable nucleic acids to pass through a three-dimensional network structure formed in the gel, and a gel material and the concentration thereof are not particularly limited. As the material of the gel filter 37, for example, an agarose or a polyacrylamide may be mentioned.

Although a thickness t of the gel filter 37 may be appropriately designed in accordance with the concentrations of proteins and nucleic acids contained in the specimen and the like, for example, the thickness t is preferably 1 to 50 mm. When the thickness t of the gel filter 37 is excessively small, the separation of nucleic acids from proteins may not be sufficiently performed. On the other hand, when the thickness t of the gel filter 37 is excessively large, a time necessary for nucleic acids to reach the recovery portion 33 after passing through the gel filter 37 is increased, and hence a time necessary for the preparation of nucleic acids is increased.

The pH of the gel filter 37 may be appropriately selected in accordance with the types of proteins contained in the specimen and the like. In addition, when an organism-derived specimen is prepared by the preparation apparatus A11, by the reason which will be described later, the gel filter 37 is preferably prepared to have a pH of 7 to 10.

### <Porous Film>

In the preparation apparatus A11 according to the first embodiment of the present disclosure, at least two porous films (the first porous film 31 and the second porous film 32) are provided in the space 3 at the anode 21 side and the cathode 22a side, respectively. The first porous film 31 and the second porous film 32 are configured to prevent a further transfer of nucleic acids and proteins received in the recovery portion 33 or the introduction portion 34 to the anode 21 side or the cathode 22a side.

As materials of the first porous film 31 and the second porous film 32, common materials, such as a cellulose acetate, a regenerated cellulose, and a polycarbonate, may be appropriately selected. Furthermore, as the porous film, an ion exchange film may also be used. In addition, the material of the first porous film 31 may be identical to or different from that of the second porous film 32, and porous films which are formed from different materials or which have different properties may be selectively used for the first porous film 31 and the second porous film 32.

An average porous diameter of the first porous film 31 is preferably 1 to 10 nm. When the average porous diameter of the first porous film 31 is set in the above range, nucleic acids present in the space E₂ of the recovery portion 33 is prevented from reaching the anode 21 by the migration thereto, and as a result, the concentration of the nucleic acids in the space E₂ of the recovery portion 33 is not decreased.

### <Anode and Cathode>

The anode 21 and the cathode 22a provided in the preparation apparatus A11 according to the first embodiment of the present disclosure are configured to generate an electric field in the space 3 (the recovery portion 33, the introduction portion 34, and the gel filter 37) sandwiched between the anode 21 and the cathode 22a. As materials of the anode 21 and the cathode 22a, common materials used for electrodes may be used. As the materials, for example, a metal, such as gold or platinum, iridium oxide, titanium nitride, and stainless steel may be mentioned.

### (3) A Method for Preparing Nucleic Acids According to an Embodiment of the Present Disclosure.

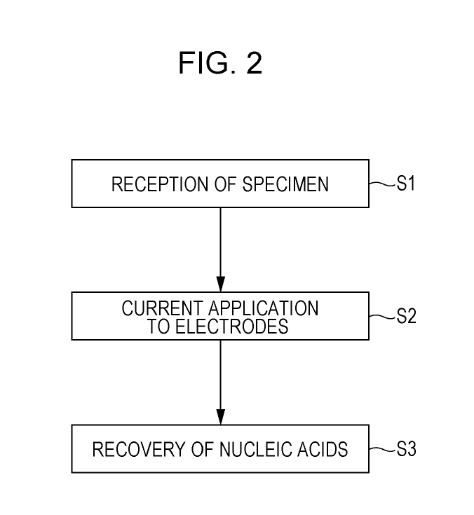

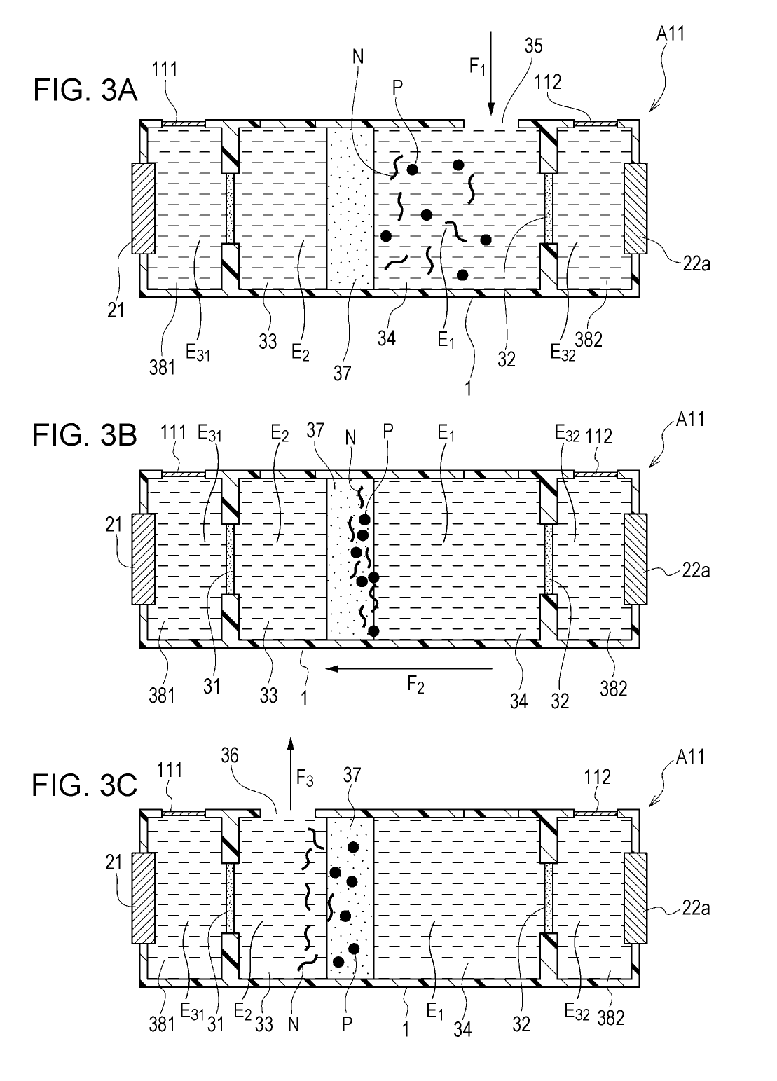

A method (hereinafter simply referred to as “preparation method” in some cases) for preparing nucleic acids according to an embodiment of the present disclosure using the above preparation apparatus A11 will be described with reference to FIGS. 2 and 3A to 3C. FIG. 2 is a flowchart showing the preparation method according to an embodiment of the present disclosure.

As shown in FIG. 2, the preparation method according to an embodiment of the present disclosure includes a step S1 of receiving a specimen containing proteins and nucleic acids in a region (introduction portion 34) sandwiched between the cathode 22a and the gel filter 37, the region being located in a space which is formed between the anode 21 and the cathode 22a and in which the gel filter 37 is provided, a step S2 of applying a current to the anode 21 and the cathode 22a, and a step S3 of recovering nucleic acids from a region (recovery portion 33) sandwiched between the gel filter 37 and the anode 21.

FIGS. 3A to 3C are schematic views each showing the behavior of nucleic acids N and proteins P in the preparation apparatus A11 in the step of the flowchart shown in FIG. 2. In addition, the preparation apparatus A11 shown in each of FIGS. 3A to 3c is a schematic cross-sectional view similar to that shown in FIG. 1.

### <Step of Receiving Specimen>

In the step S1 (step of receiving a specimen) of receiving a specimen containing proteins and nucleic acids shown in FIG. 2, the specimen containing proteins and nucleic acids is introduced into the introduction portion 34 through the inlet port 35 (FIG. 3A, see the arrow F₁). As shown in FIG. 3A, in the space E₁ of the introduction portion 34 in which the specimen is received, the nucleic acids N and the proteins P are present in the form of a mixture.

In addition, when the specimen is derived from an organism, in the method for preparing nucleic acids according to an embodiment of the present disclosure, before the step S1 is performed, a step of preparing the specimen to have a pH of 7 to 10 is preferably performed. For example, albumin (isoelectric point: 4.7) and γ-globulin (isoelectric point: 7.3), which are proteins to be contained in a large amount in an organism-derived specimen, are positively charged in an acidic solution. Hence, the proteins P described above are liable to be adsorbed to the nucleic acids N, which are negatively charged, and in the step S2 of applying a current to the anode 21 and the cathode 22a, the mobility of the nucleic acids N toward the anode 21 side is decreased.

In addition, the pH of the gel filter 37 is preferably prepared to be approximately equivalent to the pH of the specimen. Since the difference in pH between the gel filter 37 and the specimen is small, when the nucleic acids N and the proteins P migrate in the step S2 of applying a current to the anode 21 and the cathode 22a, the migration of the nucleic acids N and that of the proteins P are stabilized. That is, in the method for preparing nucleic acids according to an embodiment of the present disclosure, when an organism-derived specimen is used, a gel filter prepared to have a pH of 7 to 10 is preferably used.

After the specimen is received in the introduction portion 34, the introduction portion 34 is preferably sealed by closing the inlet port 35 with a lid or the like (in FIG. 3A, the lid is not shown). When the introduction portion 34 is sealed, the specimen is prevented from spilling out of the preparation apparatus A11. In addition, the specimen is also prevented from being contaminated. In addition, in order to prevent the inside of the recovery portion 33, for example, from being contaminated, the recovery portion 33 is also preferably sealed by closing the recovery port 36 with a lid or the like until the step S3 of recovering the nucleic acids from the region sandwiched between the gel filter 37 and the anode 21.

### <Step of Applying Current to Electrodes>

In the step S2 (step of applying a current to electrodes) of applying a current to the anode 21 and the cathode 22a shown in FIG. 2, a current is applied to the anode 21 and the cathode 22a provided in the preparation apparatus A11, and an electric field is generated in the space 3 located between the anode 21 and the cathode 22a.

As shown in FIG. 3A, when the step S2 is started, in the recovery portion 33 and the buffer solution receiving portions 381 and 382, a buffer solution having a composition suitable, for example, for the pH of the gel filter 37 is received.

When an electric field is generated in the space 3, since being negatively charged, the nucleic acids N received in the introduction portion 34 migrate toward the anode 21 side and travel in the direction toward the gel filter 37 (FIG. 3B, see the arrow F₂). On the other hand, in accordance with the pH of the specimen and the isoelectric points of the individual proteins P, the proteins P each migrate toward the anode 21 side or the cathode 22a side.

For example, when the protein P indicates albumin (isoelectric point: 4.7), and the specimen and the gel filter 37 each have a pH of 8.8, if an electric field is generated in the space 3, since being negatively charged, albumin migrates toward the anode 21 side.

As shown in FIG. 3B, by the step S2, the nucleic acids N and the proteins P travel in the gel filter 37 (see the arrow F₂). In this step, since the molecular weight of the nucleic acid N is smaller than that of the protein P, the traveling velocity of the nucleic acid N in the gel filter 37 is different from that of the protein P. In addition, since having a low degree of electrification, a protein having an isoelectric point similar to the pH of the gel filter 37 is not likely to migrate. As a result, the nucleic acids N are separated from the proteins P. The nucleic acids N separated from the proteins P travel in the gel and finally reach the recovery portion 33 through the gel filter 37.

In the step S2, a voltage applied to the anode 21 and the cathode 22a and a current-application time may be appropriately determined in accordance with separation conditions between the nucleic acids N and the proteins P, such as the concentration of the nucleic acids N contained in the specimen, the types of proteins P, and the thickness of the gel filter 37. The current application to the electrodes is preferably completed when most of the nucleic acids N reach the recovery portion 33, and when most of the proteins P are allowed to stay in the gel filter 37.

### <Step of Recovering Nucleic Acids>

In the step S3 (step of recovering nucleic acids) of recovering nucleic acids from the region (recovery portion 33) sandwiched between the gel filter 37 and the anode 21, the nucleic acids which reach the recovery portion 33 is transferred to another container to be used for analysis. In description of the step S3, for the sake of convenience, a liquid in the recovery portion 33 containing nucleic acids passing through the gel filter 37 is called a recovered liquid. The recovered liquid can be transferred to another container by introducing a pipette or the like into the recovery portion 33 through the recovery port 36 (FIG. 3C, see the arrow F₃).

By the step S2 of applying a current to the electrodes described above, the nucleic acids N reach the recovery portion 33. Since the volume of the recovery portion 33 is smaller than that of the introduction portion 34, compared to the volume of the specimen introduced into the introduction portion 34, the volume of the recovered liquid is small (see FIG. 3C). Hence, when the amount of the nucleic acids N contained in the recovered liquid is approximately equivalent to that contained in the specimen, the concentration of the nucleic acids N is increased in the recovered liquid than that in the specimen. That is, by the method for preparing nucleic acids according to an embodiment of the present disclosure, the concentration of the nucleic acids N contained in the specimen can be increased.

In addition, by the step S2 of applying a current to the electrodes described above, the proteins P are transferred into the gel filter 37 and are allowed to stay therein. Hence, the amount of the proteins P contained in the recovered liquid is decreased as compared to that of the proteins P contained in the specimen. To increase the degree of refining of the nucleic acids N indicates to decrease the concentrations of components other than the nucleic acids N contained in the liquid containing the nucleic acids N. Accordingly, by the method for preparing nucleic acids according to an embodiment of the present disclosure, the degree of refining of the nucleic acids N contained in the specimen can be increased, and hence, the nucleic acids N can be placed in a state suitable for analysis thereof.

In the method for preparing nucleic acids according to an embodiment of the present disclosure, since the gel filter 37 is used as described above, the concentration of the nucleic acids N can be increased, and the degree of refining thereof can also be increased. For example, in the method for isolating nucleic acids disclosed in Japanese Unexamined Patent Application Publication No. 2005-95003, various types of reagents are necessarily prepared, and a carrier adsorbing nucleic acids is inevitably washed a plurality of times. On the other hand, in the method for preparing nucleic acids according to an embodiment of the present disclosure, when a current is applied to the anode 21 and the cathode 22a, the nucleic acids can be prepared in a state suitable for analysis thereof. Hence, by the method for preparing nucleic acids according to an embodiment of the present disclosure, nucleic acids in a state suitable for analysis thereof can be more easily prepared.

### <Ultrasonic Treatment of Specimen>

In the method for preparing nucleic acids according to an embodiment of the present disclosure, a step of performing an ultrasonic treatment on the specimen may also be included before the receiving step S1 is performed. This step may not be necessarily performed in the method for preparing nucleic acids according to an embodiment of the present disclosure. However, for example, in the case in which the nucleic acids N are polymers, such as nucleic genomes, if the nucleic acids N are fragmented by an ultrasonic treatment, the difference in mobility from that of the proteins P is likely to be generated in the separation performed by the gel filter 37 described above. The size of nucleic acids obtained by an ultrasonic treatment is preferably in a range of 200 to 2,000 kbps.

In addition, when the nucleic acids N are present in cells contained in the specimen as in the case of genomes of bacteria, nucleic acids are likely to be released from the cells into the specimen by destroying the cell membranes using an ultrasonic treatment, so that the concentration of the nucleic acids N contained in the specimen can be easily increased, and the refining thereof can also be easily performed.

The step of performing an ultrasonic treatment may be performed using a common ultrasonic generator. For example, a contact-type ultrasonic generator, such as a horn type ultrasonic homogenizer, may be used. In addition, a non-contact type ultrasonic generator which is not in contact with a specimen may also be used. The frequency of an ultrasonic wave may be appropriately selected in accordance with the performance of the ultrasonic generator, the properties of the specimen, and the like.

### <Desalting of Specimen>

In the method for preparing nucleic acids according to an embodiment of the present disclosure, a step of desalting the specimen may also be included before the receiving step is performed. This step may not be necessarily performed in the method for preparing nucleic acids according to an embodiment of the present disclosure. However, for example, when the specimen is derived from an organism or the like, in the step S2 of applying a current to the anode 21 and the cathode 22a described above, a voltage of approximately 100 V can be easily applied to those electrodes if the concentration of a salt contained in the specimen is decreased. Hence, the separation between the nucleic acids N and the proteins P performed by using the gel filter 37 can be completed in a shorter period of time.

The desalting step may be performed by a common method used for desalting. As the method used for desalting, for example, there may be mentioned dialysis, gel filtration, desalting using an ion exchanged resin, and the like. In particular, electrical dialysis and a method using an ion exchanged resin are preferable. In addition, in order to apply a voltage of approximately 100 V to the anode 21 and the cathode 22a, the electrical conductivity of the specimen is preferably set to 2 mS/cm or less. In addition, the desalting step may be performed using the preparation apparatus A11 described above as an electrical dialysis apparatus. In this case, after the electrical conductivity of the specimen is decreased, when a voltage to be applied to the electrodes is increased, the desalting step and the specimen receiving step S1 can be continuously performed without transferring the specimen to another container.

In addition, when the method for preparing nucleic acids according to an embodiment of the present disclosure includes the above ultrasonic treatment step and the above desalting step, after the desalting step is performed, the ultrasonic treatment step is preferably performed. The reason for this is that when being held in a cell or being in the form of a polymer, nucleic acids are more unlikely to be decomposed.

### (4) An Apparatus for Preparing Nucleic Acids According to a Modification of the First Embodiment.

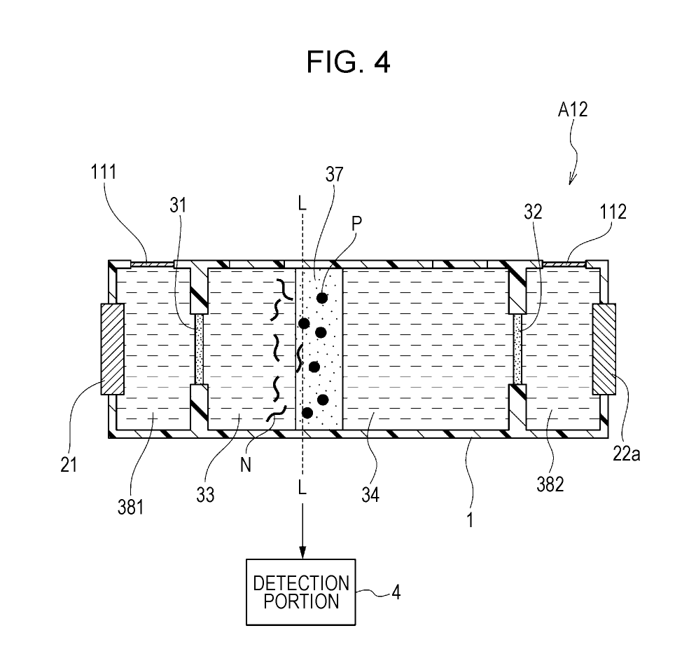

FIG. 4 is a schematic view of an apparatus for preparing nucleic acids according to a modification of the first embodiment. A preparation apparatus designated by reference numeral A12 in FIG. 4 has the same configuration as that of the first embodiment except for a detection portion 4. The same constituents as those of the first embodiment are designated by the same reference numerals as those described above, and duplicated description is omitted.

In the apparatus A12 for preparing nucleic acids, as shown in FIG. 4, there is provided the detection portion 4 configured to detect the transfer of the proteins P to a boundary L of the gel filter 37 to the recovery portion 33 (first region). As described above, in the preparation of nucleic acids using the preparation apparatus A12, before the proteins P transferred into the gel filter 37 reach the recovery portion 33 through the gel filter 37, a current application to the electrodes is preferably completed. Since detecting the arrival of the proteins P at the boundary L, the detection portion 4 is able to inform a user of an appropriate time to complete the current application. In addition, the detection portion 4 may also be configured to display the arrival of the proteins P to a user.

The detection of proteins by the detection portion 4 may be performed, for example, in an optical way. For example, when the specimen is derived from an organism, albumin bonded with dyes, such as heme and bilirubin which is a metabolic product thereof, may be contained in many cases. In addition, in blood plasma, for example, hemoglobin may also be contained in some cases. When detecting light having a specific wavelength derived from the dyes mentioned above (FIG. 4, see the arrow), the detection portion 4 can detect the arrival of the proteins P at the boundary L. In addition, a specific protein contained in the specimen may be labeled in advance with a dye besides the dyes derived from the specimen so that the detection portion 4 is able to detect the labeled protein P.

In the apparatus A12 for preparing nucleic acids according to the modification of the first embodiment, since the detection portion 4 configured to detect the arrival of the proteins P at the boundary L is provided, the current application to the electrodes can be more easily completed when the preparation of nucleic acids are suitably performed. Hence, the preparation of nucleic acids performed using the apparatus A12 for preparing nucleic acids can be more easily performed. The other effects of the preparation apparatus A12 are similar to those of the preparation apparatus A11 according to the first embodiment.

### (5) An Apparatus for Preparing Nucleic Acids According to a Second Embodiment of the Present Disclosure.

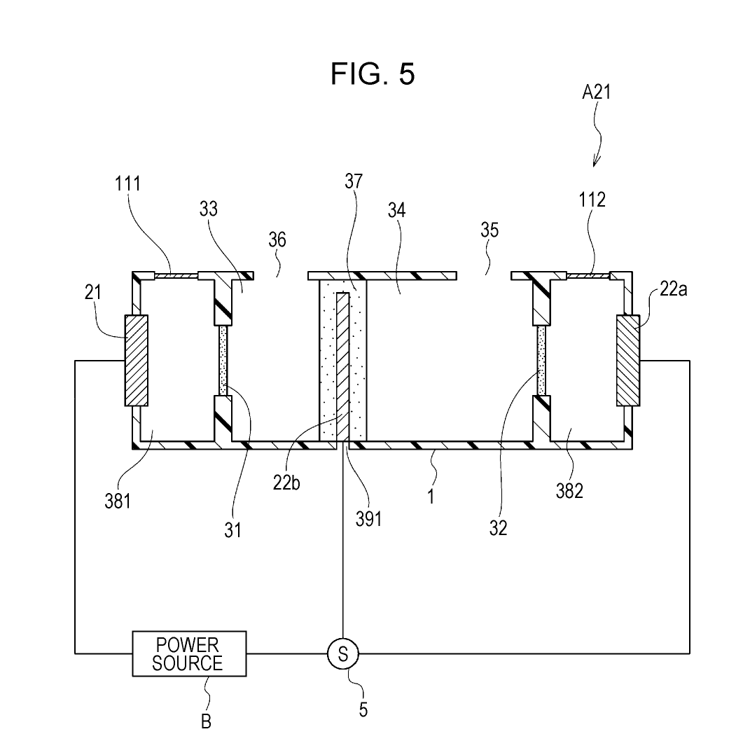

FIG. 5 is a schematic view of an apparatus for preparing nucleic acids according to a second embodiment of the present disclosure. A preparation apparatus designated by reference numeral A21 in FIG. 5 has the same configuration as that of the first embodiment except for a second cathode 22b, a first opening 391, and a switch 5. The same constituents as those in the first embodiment are designated by the same reference numerals as those described above, and duplicated description is omitted.

### <Second Cathode>

In the apparatus A21 for preparing nucleic acids according to the second embodiment, the cathode 22b may be provided in the gel filter 37 besides the cathode 22a. In description of the apparatus A21 for preparing nucleic acids, for the sake of convenience, the cathode 22a is called the first cathode 22a, and the cathode 22b provided in the gel filter 37 is called the second cathode 22b.

As described above, in the preparation of nucleic acids using the preparation apparatus A21, the current application to the electrodes is preferably completed while the proteins P are allowed to stay in the gel filter 37. On the other hand, the nucleic acids N which reach the recovery portion 33 are preferably allowed to further migrate to the anode 21 side so as to be collected on the first porous film 31; hence, the concentration of the nucleic acids N can be more increased (in FIG. 5, the nucleic acids N and the proteins P are not shown).

When the current application is performed to the anode 21 and the first cathode 22a, the second cathode 22b is polarized if provided in the gel filter 37, and an electrode reaction unexpected by a user may occur, so that the separation between the proteins P and the nucleic acids N by the gel filter 37 may not be sufficiently performed in some cases. Accordingly, the preparation apparatus A21 is configured so that the second cathode 22b to collect the nucleic acids N on the first porous film 31 can be provided in the gel filter 37 after the current application to the anode 21 and the cathode 22a is completed.

When the nucleic acids N reach the recovery portion 33, and when the proteins P are allowed to stay in the gel filter 37, the current application to the first cathode 22a is completed. Subsequently, a current application is performed to the second cathode 22b provided in the gel filter 37 and the anode 21. As a result, an electric field is generated between the second cathode 22b and the anode 21, and hence the nucleic acids N in the recovery portion 33 are collected on the first porous film 31. On the other hand, since being unlikely to be influenced by the electric field, the proteins P in the gel filter 37 are allowed to stay therein.

### <First Opening>

In the apparatus A21 for preparing nucleic acids according to the second embodiment, the configuration in which the second cathode 22b is provided in the gel filter 37 besides the first cathode 22a is not particularly limited as long as the second cathode 22b can be provided. FIG. 5 shows one example in which the first opening 391 is provided in the housing 1 so that the second cathode 22b is inserted therein. As shown in FIG. 5, when the second cathode 22b is inserted through the first opening 391, the second cathode 22b can be provided in the gel filter 37.

### <Switch>

In the apparatus A21 for preparing nucleic acids according to the second embodiment, the current application to the cathode 22b (second cathode) in the gel filter 37 and the anode 21 can be switched to and from the current application to the cathode 22a (first cathode) provided outside the gel filter 37 and the anode 21. For example, when a switch 5 is provided on wires connecting the electrodes and a power source B configured to apply a current to the anode 21, the first cathode 22a, and the second cathode 22b, the current application can be switched from the first cathode 22a to the second cathode 22b.

In the apparatus A21 for preparing nucleic acids according to the second embodiment of the present disclosure, since the second cathode 22b is provided in the gel filter 37, while the proteins P are allowed to stay in the gel filter 37, the nucleic acids N in the recovery portion 33 can be collected on the first porous film 31. Hence, in the preparation of nucleic acids using the apparatus A21 for preparing nucleic acids, the concentration of the nucleic acids N in a liquid obtained after the preparation can be increased without decreasing the degree of refining. The other effects of the preparation apparatus A21 are similar to those of the preparation apparatus A11 according to the first embodiment.

In addition, when the apparatus A21 for preparing nucleic acids has a control portion configured to control the current application to the anode 21 and the cathode 22a, the method for preparing nucleic acids according to an embodiment of the present disclosure may also be a method for preparing nucleic acids including a step of receiving a specimen containing the proteins P and the nucleic acids N in the region (introduction portion 34) sandwiched between the cathode 22a and the gel filter 37, the region being located in the space 3 which is formed between the anode 21 and the cathode 22a and in which the gel filter 37 is provided, a step of applying a current to the anode 21 and the cathode 22a by the control portion, and a step of allowing nucleic acids to migrate to the region sandwiched between the gel filter 37 and the anode 21 through the gel filter 37.

### (6) An Apparatus for Preparing Nucleic Acids According to a Modification of the Second Embodiment.

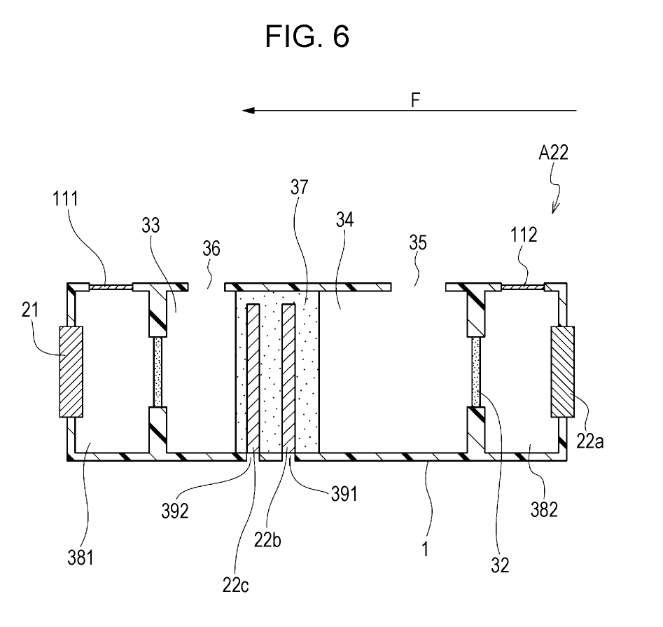

FIG. 6 is a schematic view of an apparatus for preparing nucleic acids according to a modification of the second embodiment. A preparation apparatus designated by reference numeral A22 in FIG. 6 has the same configuration as that of the second embodiment except for a third cathode 22c and a second opening 392. The same constituents as those in the second embodiment are designated by the same reference numerals as those described above, and description thereof is omitted (in addition, in FIG. 6, the power source B and the switch 5 are omitted).

### <Third Cathode>

In the apparatus A22 for preparing nucleic acids according to the modification of the second embodiment, a plurality of cathodes, the cathodes 22b and the cathode 22c, in the gel filter 37 may be provided along a transfer direction of the nucleic acids N. As shown in FIG. 6, in the preparation apparatus A22, the cathodes (the second cathode 22b and the third cathode 22c) may be provided in this order toward the anode 21 side (see the arrow F) in accordance with the transfer of the nucleic acids N in the gel filter 37. In order to provide the second cathode 22b and the third cathode 22c in the gel filter 37, as in the case of the preparation apparatus A21, for example, after the first opening 391 and the second opening 392 are provided in the housing 1, the second cathode 22b and the third cathode 22c are inserted into the gel filter 37 through the respective openings described above.

In the apparatus A22 for preparing nucleic acids according to the modification of the second embodiment, the cathode to which a current is applied may be sequentially changed from the first cathode 22a to the cathode at the anode 21 side (see the arrow F). Hence, when the transfer of the nucleic acids N into the gel filter 37 is completed, if a current application to the first cathode 22a is changed to a current application to the second cathode 22b, the amount of the proteins P to be transferred to the gel filter 37 can be decreased (in FIG. 6, the nucleic acids N and the proteins P are not shown). Furthermore, when the nucleic acids N reach the recovery portion 33, if a current application to the second cathode 22b is changed to a current application to the third cathode 22c, the nucleic acids N can be collected on the first porous film 31 while the proteins P is allowed to stay in the gel filter 37.

In addition, in FIG. 6, although the case in which the two cathodes, that is, the second cathode 22b and the third cathode 22c, are provided is shown by way of example, in the preparation apparatus A22 according to the modification of the second embodiment, the number of cathodes to be provided is not particularly limited, and at least three cathodes may also be provided.

In the apparatus A22 for preparing nucleic acids according to the modification of the second embodiment, a plurality of cathodes (the second cathode 22b and the third cathode 22c) may be provide in the gel filter 37. Hence, in the preparation of nucleic acids using the preparation apparatus A22, the concentration of nucleic acids can be increased while the proteins P are further prevented from being mixed therewith. Accordingly, in the preparation of nucleic acids using the apparatus A22 for preparing nucleic acids, the concentration of the nucleic acids N in a liquid to be obtained after the preparation can be increased without decreasing the degree of refining. The other effects of the preparation apparatus A22 are similar to those of the preparation apparatus A11 according to the first embodiment.

In addition, according to an embodiment of the present disclosure, the following configurations may also be formed.

(1) An apparatus for preparing nucleic acids which includes an anode, a cathode, and a space formed between the anode and the cathode and in which the space includes a first porous film provided at an anode side, a second porous film provided at a cathode side, an inlet port provided in a region sandwiched between the first porous film and the second porous film to introduce a specimen containing proteins and nucleic acids into the region, and a gel filter provided between the inlet port and the first porous film.
(2) The apparatus for preparing nucleic acids of the above (1) in which the volume of a first region defined by the gel filter and the first porous film is small as compared to that of a second region defined by the gel filter and the second porous film.
(3) The apparatus for preparing nucleic acids of the above (1) or (2) in which an average porous diameter of the first porous film is 1 to 10 nm.
(4) The apparatus for preparing nucleic acids of one of the above (1) to (3) in which the gel filter is prepared to have a pH of 7 to 10.
(5) The apparatus for preparing nucleic acids of one of the above (1) to (4) which further includes a detection portion configured to detect a transfer of the proteins to a boundary of the gel filter to the first region.
(6) The apparatus for preparing nucleic acids of one of the above (1) to (5) which is used to prepare a template nucleic acid of a nucleic acid amplification reaction.
(7) The apparatus for preparing nucleic acids of one of the above (1) to (6) in which besides the cathode, another cathode may also be provided in the gel filter.
(8) The apparatus for preparing nucleic acids of the above (7) in which the number of the cathodes provided in the gel filter may be at least two along a transfer direction of the nucleic acids.
(9) The apparatus for preparing nucleic acids of the above (7) or (8) in which a current application to the anode and the cathode in the gel filter may be switched to and from a current application to the anode and the cathode provided outside the gel filter.
(10) A method for preparing nucleic acids including a step of receiving a specimen containing proteins and nucleic acids in a region sandwiched between a cathode and a gel filter, the region being located in a space which is formed between an anode and the cathode and in which the gel filter is provided, a step of applying a current to the anode and the cathode, and a step of allowing the nucleic acids to migrate to a region sandwiched between the anode and the gel filter through the gel filter.
(11) A method for preparing nucleic acids including a step of receiving a specimen containing proteins and nucleic acids in a region sandwiched between a cathode and a gel filter, the region being located in a space which is formed between an anode and the cathode and in which the gel filter is provided, a step of applying a current to the anode and the cathode, and a step of recovering the nucleic acids from a region sandwiched between the gel filter and the anode.
(12) The method for preparing nucleic acids of the above (11) in which the gel filter is prepared to have a pH of 7 to 10.
(13) The method for preparing nucleic acids of the above (11) or (12) which further includes a step of performing an ultrasonic treatment on the specimen before the receiving step is performed.
(14) The method for preparing nucleic acids of one of the above (11) to (13) which further includes a step of desalting the specimen before the receiving step is performed.
(15) The method for preparing nucleic acids of one of the above (10) to (14) which prepares a template nucleic acid of a nucleic acid amplification reaction.
(16) The method for preparing nucleic acids of the above (15) in which the nucleic acid amplification reaction is performed by a polymerase chain reaction (PCR) method.
(17) The method for preparing nucleic acids of the above (15) in which the nucleic acid amplification reaction is an isothermal nucleic acid amplification reaction.

### EXAMPLES

### Experimental Example 1

### 1. Increase in Concentration of Nucleic Acids in a Specimen Using an Apparatus for Preparing Nucleic Acids According to an Embodiment of the Present Disclosure

By preparing a specimen containing nucleic acids and proteins using an apparatus for preparing nucleic acids according to an embodiment of the present disclosure, it was investigated whether the concentration of the nucleic acids contained in the specimen could be increased or not. In addition, by using the preparation apparatus described above, it was also investigated whether the concentration of the proteins contained in the specimen could be decreased or not and whether the degree of refining of the nucleic acids could be increased.

### (Material and Method)

A nucleic acid chain having a length of 20 bps and modified with a fluorescent dye Cy3 was added to desalted bovine blood plasma to prepare a specimen of this experimental example. In addition, in this experimental example, the specimen was prepared using the preparation apparatus A11 shown in FIG. 1. The details of the preparation apparatus A11 are as follows.

As the first porous film 31 and the second porous film 32, there was used a dialysis membrane having an average pore diameter of 5 nm and washed with sodium carbonate. For the gel filter 37, there was used a 0.5% agarose gel (seakem gold agarose, manufactured by CAMBREX) formed by using 1×TBE (0.089 M Tris-borate, 0.089 M Boric Acid, 0.002 M EDTA) prepared to have a pH of 8.8. In addition, 1×TBE (pH: 8.8) was filled in each of the buffer solution receiving portions 381 and 382 and the recovery portion 33.

After the above specimen was received in the introduction portion 34, a current was applied to the anode 21 and the cathode 22a at 100 V. After the current application was started, 20 μl of a liquid was recovered from the recovery portion 33 every 10 minutes. The concentration of nucleic acid chains and that of proteins contained in the recovered liquid were measured using an UV visible spectrophotometer (Nanodrop ND-1000, manufactured by NanoDrop Technologies Inc.). The quantitative determination of nucleic acids was performed by calculation from a fluorescence intensity at a wavelength of 570 nm, and the quantitative determination of proteins was performed by calculation from an absorbance at a wavelength of 260 nm.

### (Results)

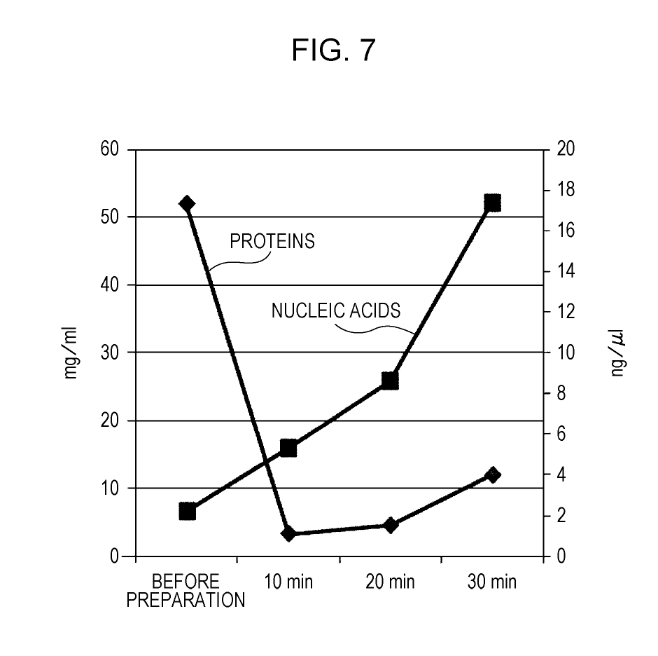

The results of this experimental example are shown in FIG. 7. The vertical axis at the right side shown in FIG. 7 indicates the concentration of nucleic acids, and the vertical axis at the left side indicates the concentration of proteins. As shown in FIG. 7, compared to the concentration of the nucleic acids of the specimen before the preparation was performed, the concentrations of nucleic acids contained in the liquids recovered at 10, 20, and 30 minutes from the start of the current application were increased. From the results described above, it was confirmed that when a specimen containing nucleic acids was prepared using the preparation apparatus according to an embodiment of the present disclosure, the concentration of the nucleic acids could be increased.

In addition, compared to the concentration of the proteins of the specimen before the preparation was performed, the concentrations of proteins contained in the liquids recovered at 10, 20, and 30 minutes from the start of the current application were decreased. From the results described above, it was confirmed that when a specimen containing nucleic acids and proteins was prepared using the preparation apparatus according to an embodiment of the present disclosure, the nucleic acids and the proteins were separated from each other, and the concentration of the proteins contained in the liquid could be decreased. That is, it was confirmed that in the liquid recovered from the recovery portion 33, the degree of refining of nucleic acids was increased.

### Experimental Example 2

### 2. Analysis of Nucleic Acids Contained in a Liquid Prepared by a Preparation Apparatus

It was investigated whether the nucleic acids in the specimen prepared by the preparation apparatus described above were suitable for analysis or not as compared to the nucleic acids in the specimen before the preparation was performed. In this experimental example, as an analysis method, a nucleic acid amplification reaction was selected, and amplification of nucleic acids was performed by an LAMP method.

In this experimental example, bifidobacteria were added to a porcine spinal fluid to have a concentration of 1,000 cells/μl, so that a specimen was prepared. After treated by an ultrasonic treatment and a desalting treatment, this specimen was received in the introduction portion 34 of the preparation apparatus described in Experimental Example 1, and a current was then applied to the electrodes at 100 V for 12 minutes for preparation.

After the current application to the electrodes was completed, 20 μl of a liquid was recovered from the recovery portion, and 5 μl of the liquid thus recovered was then charged into each of three tubes. Next, 5 μl of a reagent solution for an LAMP reaction was charged into each tube receiving the recovered liquid, so that test groups 1 to 3 were prepared. In addition, as control groups, instead of using the recovered liquid, 5 μl of the specimen before the preparation was performed by the preparation apparatus was charged into each tube, and 5 μl of the reagent solution for an LAMP reaction was charged into the tube, so that control groups 1 to 3 were prepared.

The test groups 1 to 3 and the control groups 1 to 3 were maintained at 60° C. using a thermal cycler (DNA Engine, manufactured by BIO-RAD), and the amplification of nucleic acids was measured for 90 minutes.

In this experimental example, in order to detect amplified nucleic acid chains, a QProbe was used. In the Qprobe, fluorescent-labeled cytosine is present at the terminal, and when the Qprobe is not hybridized with a nucleic acid chain, this fluorescent substance emits light. On the other hand, when the Qprobe is hybridized with a nucleic acid chain, the fluorescent-labeled cytosine faces guanine, and as a result, the fluorescent substance is quenched due to the influence of guanine. Hence, when a Qprobe to be hybridized with an amplified nucleic acid chain is used in a nucleic acid amplification reaction, the amplification of nucleic acids can be detected by the decrease in fluorescence intensity derived from the Qprobe.

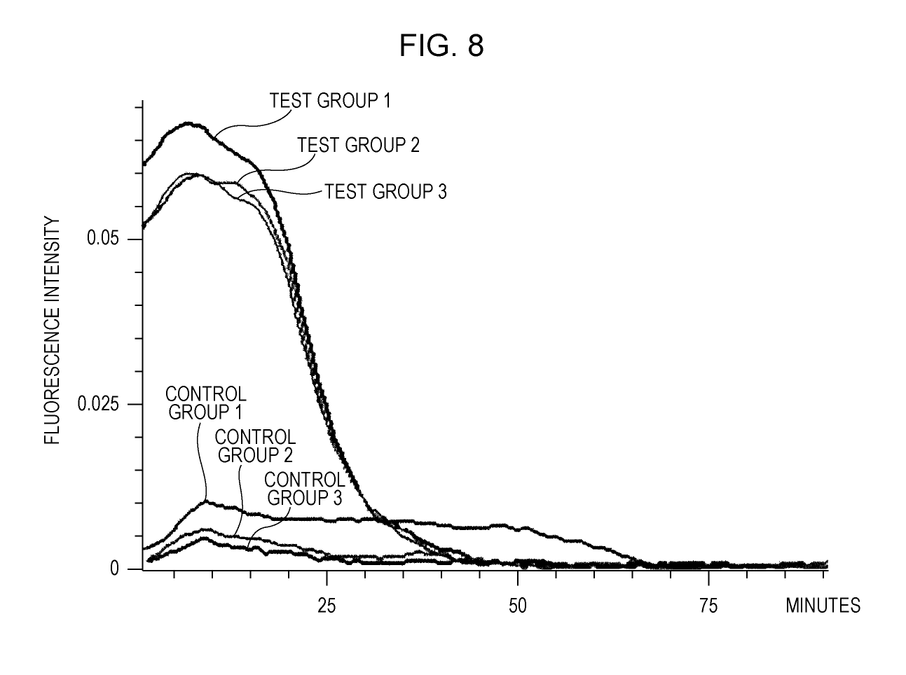

The results of this experimental example are shown in FIG. 8. In the test groups 1 to 3, the fluorescence intensity decreased with time, and hence, it was confirmed that the nucleic acids were amplified. On the other hand, in the control groups 1 to 3, the decrease in fluorescence intensity was not observed unlike the test groups 1 to 3, and hence, it was confirmed that the nucleic acids were not amplified.

From the results of this experimental example, when a specimen containing nucleic acids and proteins is prepared using the preparation apparatus according to an embodiment of the present disclosure, it is confirmed that the specimen can be placed in a state suitable for analysis of the nucleic acids.

It should be understood by those skilled in the art that various modifications, combinations, sub-combinations and alterations may occur depending on design requirements and other factors insofar as they are within the scope of the appended claims or the equivalents thereof.

What is claimed is:

1. An apparatus for preparing nucleic acids comprising:
an anode;
a cathode; and
a space formed between the anode and the cathode,
wherein the space includes
a first porous film provided at an anode side,
a second porous film provided at a cathode side,
an inlet port which is provided in a region sandwiched between the first porous film and the second porous film and which introduces a specimen containing proteins and nucleic acids into the region, and
a gel filter provided between the inlet port and the first porous film.

2. The apparatus for preparing nucleic acids according to claim 1,
wherein the gel filter and the first porous film define a first region,
the gel filter and the second porous film define a second region, and
the volume of the first region is small as compared to the volume of the second region.

3. The apparatus for preparing nucleic acids according to claim 1,
wherein the first porous film has an average porous diameter of 1 to 10 nm.

4. The apparatus for preparing nucleic acids according to claim 1,
wherein the gel filter is prepared to have a pH of 7 to 10.

5. The apparatus for preparing nucleic acids according to claim 1,
wherein the gel filter and the first porous film define a first region,
further comprising a detection portion which detects a transfer of the proteins to a boundary of the gel filter to the first region.

6. The apparatus for preparing nucleic acids according to claim 1,
wherein the apparatus for preparing nucleic acids is used to prepare a template nucleic acid of a nucleic acid amplification reaction.

7. The apparatus for preparing nucleic acids according to claim 1,
further comprising, besides the cathode, at least one another cathode in the gel filter.

8. The apparatus for preparing nucleic acids according to claim 7,
wherein the number of the cathodes in the gel filter is at least two, and
the cathodes in the gel filter are provided along a transfer direction of the nucleic acids.

9. The apparatus for preparing nucleic acids according to claim 7,
wherein a current application to the anode and the cathode provided in the gel filter is switched to and from a current application to the anode and the cathode provided outside the gel filter.

10. A method for preparing nucleic acids comprising:
receiving a specimen containing proteins and nucleic acids in a region sandwiched between a cathode and a gel filter, the region being located in a space which is formed between the cathode and an anode and in which the gel filter is provided;
applying a current to the anode and the cathode; and
allowing the nucleic acids to migrate to a region sandwiched between the anode and the gel filter through the gel filter.

11. A method for preparing nucleic acids comprising:
receiving a specimen containing proteins and nucleic acids in a region sandwiched between a cathode and a gel filter, the region being located in a space which is formed between the cathode and an anode and in which the gel filter is provided;
applying a current to the anode and the cathode; and
recovering the nucleic acids from a region sandwiched between the anode and the gel filter.

12. The method for preparing nucleic acids according to claim 11,
wherein the gel filter is prepared to have a pH of 7 to 10.

13. The method for preparing nucleic acids according to claim 11,
further comprising, before the receiving a specimen, performing an ultrasonic treatment on the specimen.

14. The method for preparing nucleic acids according to claim 11,
further comprising, before the receiving a specimen, desalting the specimen.

15. The method for preparing nucleic acids according to claim 10,
wherein the method for preparing nucleic acids prepares a template nucleic acid of a nucleic acid amplification reaction.

16. The method for preparing nucleic acids according to claim 15,
wherein the nucleic acid amplification reaction is performed by a polymerase chain reaction (PCR) method.

17. The method for preparing nucleic acids according to claim 15,
wherein the nucleic acid amplification reaction is an isothermal nucleic acid amplification reaction.
